(19) United States
(12) Patent Application Publication
(10) Pub. No.: US 2026/0045330 A1
(43) Pub. Date: Feb. 12, 2026

(54) VIRTUAL HEALTHCARE ENVIRONMENT

(71) Applicant: PROGNOSTIC OPTIMIZATION GROUP INC., Euless, TX (US)

(72) Inventors: Jayson Raymond Bakonyi, Euless, TX (US); Raymond H. Chen, San Marino, CA (US); Ramaswamy N. Melkote, Los Altos, CA (US); Igor Novikov, Mountain View, CA (US); Marc Teitelbaum, Hoboken, NJ (US)

(73) Assignee: PROGNOSTIC OPTIMIZATION GROUP INC., Euless, TX (US)

(21) Appl. No.: 19/291,764

(22) Filed: Aug. 6, 2025

Related U.S. Application Data
(60) Provisional application No. 63/680,873, filed on Aug. 8, 2024.

(51) Int. Cl. G16H 40/20; G16H 10/60; G16H 15/00; G16H 20/00; G16H 50/20

(52) U.S. Cl. CPC G16H 10/60; G16H 15/00; G16H 50/20

(57) ABSTRACT

Apparatus and associated methods relate to an automatic concierge of a virtual health system (VHS). In an illustrative example, a VHS may include a triage engine. The triage engine may be configured to receive information, including insurance information, location information, and/or symptoms from a remote patient. The triage engine may apply the received information to an artificial intelligence risk assessment model (AIRAM). The AIRAM may determine a risk assessment of the remote patient and a recommendation based on the received input. In some implementations, the AIRAM may recommend the remote patient to seek immediate treatment when the assessed risk is higher than a predetermined threshold. In some examples, the AIRAM may generate a physical preparation report (PPR). For example, the PPR may include treatment recommendations based on the received input for the physician's considerations. Various embodiments may advantageously enhance virtual healthcare efficiency and reduce waiting time for virtual healthcare appointments.

### CROSS-REFERENCE TO RELATED APPLICATIONS

This application is a non-provisional application and claims the benefit of U.S. Application Ser. No. 63/680,873, titled “Virtual Healthcare Environment,” filed by Jayson Raymond Bakonyi et al. on Aug. 8, 2024.

This application incorporates the entire contents of the foregoing application(s) herein by reference.

### TECHNICAL FIELD

Various embodiments relate generally to virtual healthcare systems.

### BACKGROUND

Healthcare is a component of society, encompassing the prevention, diagnosis, treatment, and management of illness and injury. With advancements in technology, the healthcare industry has undergone transformation, especially in the way care is delivered. Traditional in-person models are increasingly being supplemented or replaced by innovative digital solutions aimed at improving accessibility, efficiency, and patient outcomes.

Virtual healthcare, also known as telehealth or telemedicine, enables the delivery of medical services through digital platforms. Examples include video consultations between patients and providers, remote patient monitoring using wearable devices, and mobile health applications that track vital signs or medication adherence. These tools increase access to care for patients in remote or underserved areas and also help reduce the strain on healthcare systems by streamlining administrative tasks and improving care coordination.

### SUMMARY

Apparatus and associated methods relate to an automatic concierge of a virtual health system (VHS). In an illustrative example, a VHS may include a triage engine. The triage engine, for example, may be configured to receive information, including insurance information, location information, and/or symptoms from a remote patient. For example, the triage engine may apply the received information to an artificial intelligence risk assessment model (AIRAM). The AIRAM, for example, may determine a risk assessment of the remote patient and a recommendation based on the received input. In some implementations, the AIRAM may recommend the remote patient to seek immediate treatment when the assessed risk is higher than a predetermined threshold. In some examples, the AIRAM 125 may also generate a physical preparation report (PPR). For example, the PPR may include treatment recommendations based on the received input for the physician's considerations. Various embodiments may advantageously enhance virtual healthcare efficiency and reduce waiting time for a virtual healthcare appointment.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary virtual healthcare system (VHS) employed in an illustrative use-case scenario.

FIG. 2 is a block diagram depicting an exemplary VHS.

FIG. 3 is a flowchart illustrating an exemplary virtual healthcare patient reception method.

FIG. 4 is a flowchart illustrating an exemplary two patient process flow method.

FIG. 5 is a flowchart illustrating an exemplary method of an adaptive clinical decision support system with provider diagnostic nudges.

FIG. 6 is a flowchart illustrating an exemplary comorbidity agent method.

Like reference symbols in the various drawings indicate like elements.

### DETAILED DESCRIPTION OF ILLUSTRATIVE EMBODIMENTS

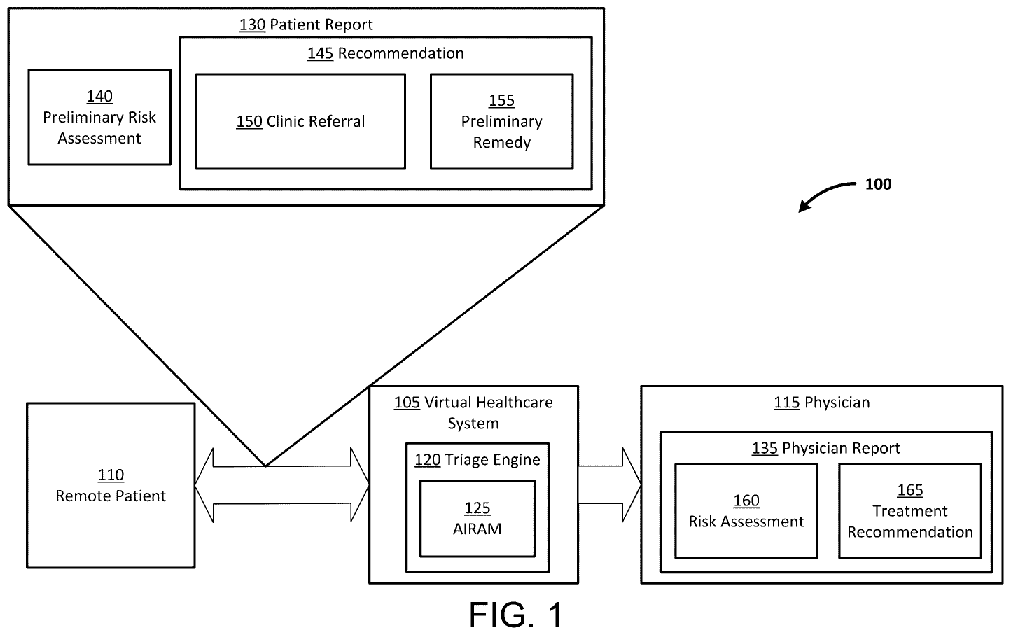

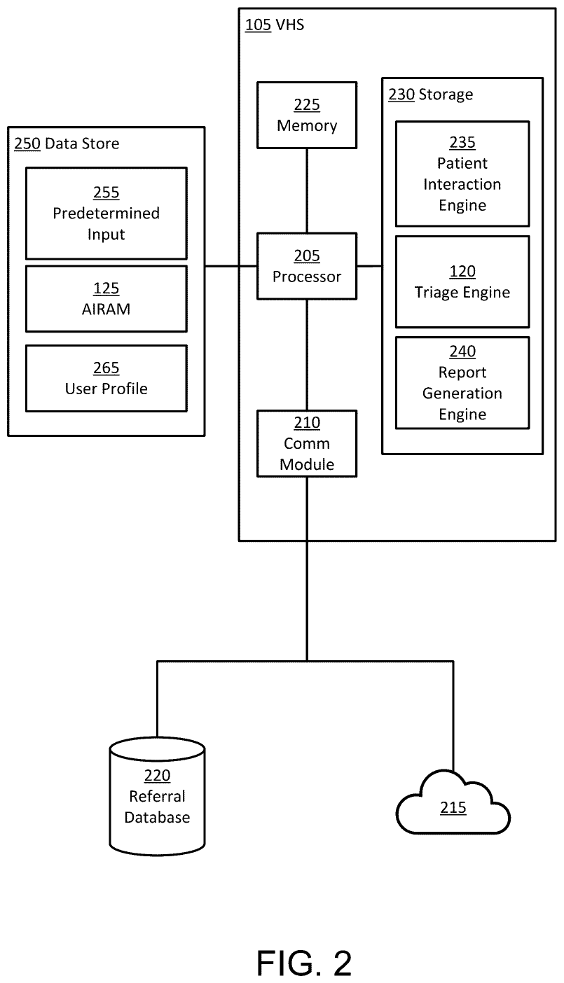

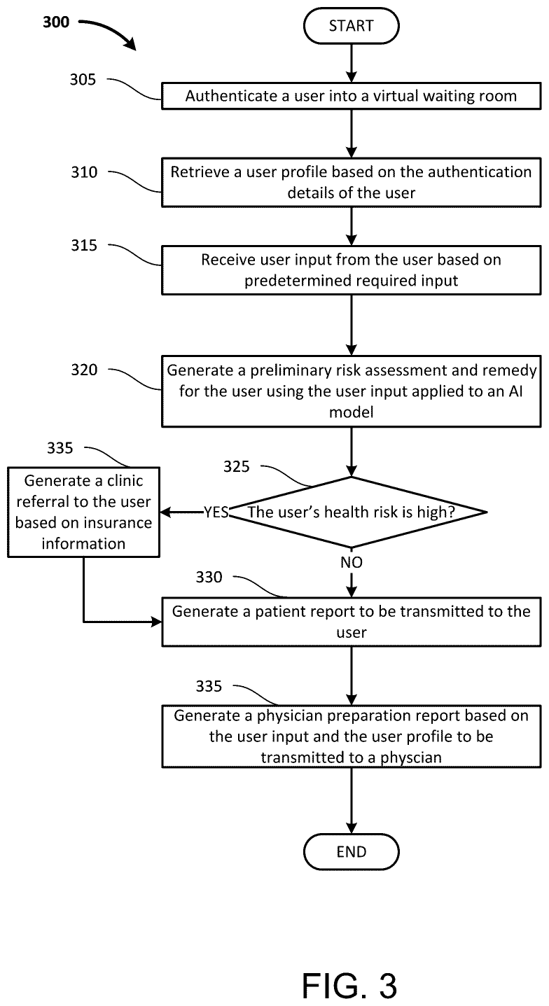

To aid understanding, this document is organized as follows. First, to help introduce discussion of various embodiments, a virtual healthcare system (VHS) is introduced with reference to FIGS. 1-2. Second, that introduction leads into a description with reference to FIGS. 3-6 of some exemplary embodiments of methods performed by the VHS. Finally, the document discusses further embodiments, exemplary applications and aspects relating to the VHS.

FIG. 1 depicts an exemplary virtual healthcare system (VHS) employed in an illustrative use-case scenario. In an exemplary scenario 100, a VHS 105 may be (virtually) connected to a remote patient 110 and a physician 115. For example, the VHS 105 may include a telemedicine environment. For example, the remote patient 110 and the physician 115 may be connected to the VHS 105 via a network (e.g., the Internet). For example, the remote patient 110 may receive a consultation service from the physician 115 virtually through the VHS 105. In some embodiments, the VHS 105 may include a web server. For example, the remote patient 110 may be required to log into the VHS 105 when it is the appointment time between the remote patient 110 and the physician 115.

In this example, the VHS 105 includes a triage engine 120 configured to communicate automatically with the remote patient 110. For example, the triage engine 120 may be configured to communicate with the remote patient 110 when the remote patient 110 logs into the VHS 105 before the appointment starts. In some implementations, the remote patient 110 may be configured to request information from the remote patient 110. For example, the triage engine 120 may include a form to be filled in by the 110. For example, the triage engine 120 may include a chatbot configured to converse with the remote patient 110 to obtain predetermined information.

In some implementations, the predetermined information may include a location of the remote patient 110. In some implementations, the predetermined information may include insurance information of the remote patient 110. In some implementations, the predetermined information may include a list of symptoms of the remote patient 110.

As shown, the triage engine 120 includes an artificial intelligence risk assessment model (AIRAM 125). For example, the triage engine 120 may be configured to process, using the AIRAM 125, input received from the remote patient 110. For example, the triage engine 120 may apply the input to the AIRAM 125 to generate a patient report 130 and a physician report 135.

In some embodiments, the triage engine 120 may generate the patient report 130 based on the predetermined information. In this example, the patient report 130 includes a preliminary risk assessment 140 and a recommendation 145. The preliminary risk assessment 140 may, for example, include a determined urgency based on the input received from the remote patient 110. For example, the determined urgency may include a rating of risk associated with the remote patient 110. In some examples, the rating of risk may include recommending the remote patient 110 to, for example, call an emergency number, and/or go to emergency rooms or clinics nearby.

The recommendation 145, for example, may include a clinic referral 150 based on the patient's geolocation, medical history, symptoms, and/or insurance information. As an illustrative example without limitation, the triage engine 120 may determine that the remote patient 110 is suffering from coronavirus disease (COVID) and recommend the remote patient 110 to a nearby COVID testing center based on the remote patient 110's geolocation. In some examples, the recommendation 145 may also include preliminary remedies 155 (e.g., for relieving certain symptoms) suggested to the remote patient 110.

In some implementations, the physician 115 may receive the physician report 135 from the triage engine 120 for preparation of the virtual appointment with the remote patient 110 (e.g., while the remote patient 110 is in a virtual waiting room before meeting the physician 115). The physician report 135 includes a risk assessment 160 generated based on the patient's symptoms and medical history. For example, the physician 115 may gauge a health risk of the remote patient 110 based on the risk assessment 160. For example, the physician 115 may adjust its strategy based on the risk assessment 160.

As shown, the physician report 135 includes a treatment recommendation 165. The treatment recommendation, for example, may include suggested prescription medication details. In some examples, the treatment recommendation 165 may include a request to the physician 115 to encourage the remote patient 110 to make a non-virtual appointment and/or to visit an emergency room immediately, based on a determined health condition of the remote patient 110 (e.g., by the AIRAM 125).

In various implementations, the VHS 105 may be configured to perform a risk assessment based on patient-provided data. For example, the preliminary risk assessment 140 may help the remote patient 110 to identify potential health risks at present and/or determine the urgency of medical intervention. For example, the AIRAM 125 may be configured to be updated by training. For example, the triage engine 120 may generate risk assessments and treatment recommendations efficiently and accurately. Various embodiments may advantageously shorten the time for a virtual appointment. In some examples, some embodiments may aid healthcare providers by summarizing patient data and suggesting possible responses, including specific treatment options and necessary referrals. In some examples, the remote patient 110 may, knowing the preliminary risk assessment 140, leave the virtual environment and proceed to an emergency room for urgent care.

In some embodiments, a triage program (e.g., the triage engine 120) of a telemedicine platform (e.g., the VHS 105) may be configured to, based on patient's remote input, generate a patient feedback report (e.g., the patient report 130) including a preliminary risk assessment analysis (e.g., the preliminary risk assessment 140) and a treatment plan (e.g., the preliminary remedies 155). In some examples, the patient report 130 may include a referral recommendation (e.g., the clinic referral 150) generated as a function of the patient's geolocation and/or insurance information. For example, the telemedicine platform may generate a physician preparation report (e.g., the physician report 135). For example, the physical preparation report may include medical risk assessment (e.g., the risk assessment 160) based on the patient's symptoms and medical history, and a suggested response to the physician to the patient (e.g., the treatment recommendation 165).

The patient report 130 may, for example, advantageously translate complex medical information into accessible insights, providing personalized treatment recommendations that are specifically phrased to maximize understanding and adherence. The patient report 130 may, for example, advantageously include easy-to-understand health metrics with visual representations and contextual explanations that help patients interpret their clinical data. The patient report 130 may, for example, advantageously incorporate preventive care suggestions tailored to the patient's specific condition and risk factors, creating a comprehensive health management tool. The patient report 130 may, for example, concludes with next steps for managing a condition, advantageously creating an actionable pathway that enhances engagement and improves outcomes.

The physician report 135 may, for example, advantageously deliver structured clinical data optimized for medical decision-making in a format designed to integrate with clinical workflows. The physician report 135 may, for example, comprehensive symptom analysis with weighted probability assessments for different interpretations of the presented symptoms. The physician report 135 may, for example, advantageously provide evidence-based differential diagnoses ranked by likelihood based on the specific combination of patient factors and symptoms. The physician report 135 may, for example, suggest treatment protocols with references to clinical guidelines and consideration of patient-specific factors that might influence treatment selection. The physician report 135 may, for example, incorporate relevant patient history contextually organized around the current presentation to facilitate efficient review.

In some implementations, the suggested response may include prescription details based on the patient's medical history and symptoms. In some implementations, the suggested response may include referral details of an emergency room and/or a specialist for treating the patient more effectively.

FIG. 2 is a block diagram depicting an exemplary VHS. The VHS 105, as shown, includes a processor 205. The processor 205 may, for example, include one or more processing units. The processor 205 is operably coupled to a communication module 210. The communication module 210 may, for example, include wired communication. The communication module 210 may, for example, include wireless communication. In the depicted example, the communication module 210 is operably coupled to a communication network 215 and a referral database 220. For example, the communication network 215 may include the Internet. For example, the communication network 215 may be configured to connect the remote patient 110 and the physician 115 to the VHS 105. The referral database 220, for example, may include clinics and specialists in an area (e.g., in the United States). For example, the referral database 220 may include insurance information and location information of the clinics.

The processor 205 is operably coupled to a memory module 225. The memory module 225 may, for example, include one or more memory modules (e.g., random-access memory (RAM)). The processor 205 includes a storage module 230. The storage module 230 may, for example, include one or more storage modules (e.g., non-volatile memory). In the depicted example, the storage module 230 includes a patient interaction engine (PIE 235), the triage engine 120, and a report generation engine (RGE 240). The PIE 235, for example, may include a web interface configured to transmit and receive information to and from the remote patient 110. In some implementations, the PIE 235 may include an (AI) chatbot. In some examples, the PIE 235 may include an interface configured to receive user credentials from the remote patient 110. In some examples, the PIE 235 may include a form for receiving the predetermined input (e.g., location, symptoms, medical history, insurance information) from the remote patient 110.

The triage engine 120, for example, may apply the received patient input from the PIE 235 to the AIRAM 125. For example, the triage engine 120 may use the AIRAM 125 to generate a risk assessment and a recommendation for the remote patient 110 based on the patient's input.

For example, the RGE 240 may generate the patient report 130 to be displayed at a patient device of the remote patient 110 and the physician report 135 to a physician device of the physician 115. For example, the patient device and the physician device may be connected to the VHS 105 via the communication network 215.

The processor 205 is further operably coupled to a data store 250. The data store 250 includes a predetermined input 255, the AIRAM 125, and a user profile 265. For example, the PIE 235 may automatically generate questions to be transmitted to the remote patient 110 based on the predetermined input 255. For example, the predetermined input 255 may include a demographic information of the remote patient 110, a geographic information of the remote patient 110, and a list of symptoms relevant to this visit of the remote patient 110.

The AIRAM 125, for example, may be configured to receive the location, the symptoms, the insurance information, and a medical history of a patient as input. For example, the medical history may be included in the user profile 265. For example, the AIRAM 125 may generate preliminary risk assessment 140 and the recommendation 145 based on the user input and the user profile 265. For example, the AIRAM 125 may generate to the physician 115 the risk assessment 160 and the treatment recommendation 165 based on the user input and the treatment recommendation 165.

The user profile 265, for example, may include demographic information of a user (e.g., the remote patient 110). For example, the user profile 265 may include age and medical history (e.g., historical test reports, historical prescriptions, previous appointments history) of the user. For example, the user profile 265 may include insurance details of the user.

FIG. 3 is a flowchart illustrating an exemplary virtual healthcare patient reception method 300. For example, the method 300 may be performed by the triage engine 120. In this example, the method 300 begins in step 305 when a user is authenticated into a virtual waiting room. For example, the triage engine 120 may verify the user's login credentials.

In step 310, a user profile is retrieved based on the authentication details of the user. For example, the triage engine 120 may access the user profile 265 from the data store 250.

In step 315, user input is received from the user based on predetermined required input. For example, the PIE 235 may prompt the user to provide information such as symptoms, medical history, and location (e.g., based on the predetermined input 255). The AIRAM 125 may, for example, generate tailored follow-up questions based on a user's input. The PIE 235 may, for example, generate questions and responses in a natural language. The PIE 235 may, for example, advantageously enable patients to describe their symptoms in a conversational, open-ended manner, similar to speaking with a physician.

In step 320, a preliminary risk assessment and remedy for the user is generated using the user input applied to an AI model. For example, the triage engine 120 may use the AIRAM 125 to analyze the user input and generate the preliminary risk assessment 140 and the recommendation 145.

At a decision point 325, it is determined whether the user's health risk is high. For example, the AIRAM 125 may evaluate the risk level based on the input data. For example, the triage engine 120 may include a predetermined risk threshold. For example, a risk higher than the predetermined risk threshold is determined to be high.

If the user's health risk is not high, in step 330, a patient report is generated to be transmitted to the user. For example, the RGE 240 may compile the preliminary risk assessment 140 and the recommendation 145 to be transmitted to the remote patient 110.

If the user's health risk is high, in step 335, a clinic referral is generated for the user based on insurance information, and the step 330 is performed. For example, the triage engine 120 may recommend nearby clinics or specialists covered by the user's insurance.

After the patient report is generated, in step 335, a physician preparation report is generated based on the user input and the user profile to be transmitted to a physician. For example, the RGE 240 may generate the physician report 135 including the risk assessment 160 and the treatment recommendation 165, and the method 300 ends.

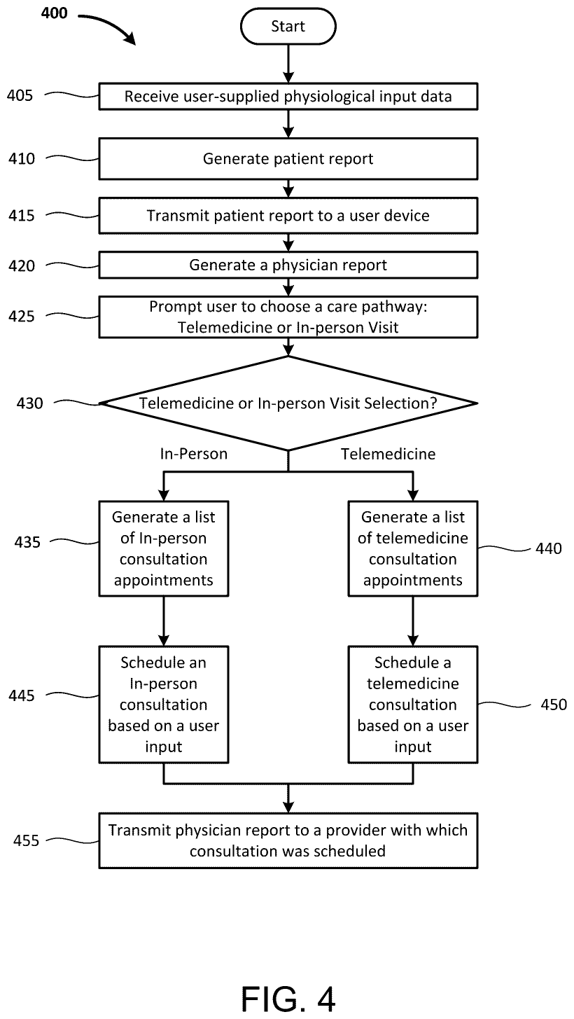

FIG. 4 is a flowchart illustrating an exemplary two patient process flow method 400. For example, the method 400 may be performed by the VHS 105. In a step 405, the VHS 105 may, for example, receive user-supplied physiological input data. For example, the VHS 105 may generate auto-filled response fields based on predetermined protocols of a biomedical data processing system and receive user-supplied physiological input data from a user's interaction with the auto-filled response fields.

In a step 410, the VHS 105 may, for example, generate a patient report (e.g., patient report 130). The patient report may, for example, include preliminary diagnosis and treatment report. The patient report may, for example, include over-the-counter options appropriate to treat a condition of the user. In a step 415, the VHS 105 may, for example, transmit the patient report to a user device of the user.

In a step 420, the VHS 105 may, for example, generate a physician report (e.g., physician report 135). The physician report may, for example, include the user's symptoms. The physician report may, for example, include the user's likely diagnosis. The physician report may, for example, include the user's recommended first-line treatment prescription options.

In a step 425, the VHS 105 may, for example, generate prompts to the user to choose a care pathway. The care pathway may, for example, include a telemedicine pathway and an in-person visit pathway. In a step 430, the VHS 105 reaches a decision point where it decides whether it has received a user-selected care pathway of in-person or telemedicine. If the VHS 105 receives a decision of an in-person care pathway, then the method 400 proceeds to a step 435. In a step 435, the VHS 105 generates a list of in-person consultation appointments with a healthcare provider. For example, the VHS 105 may consider user-reported symptoms, user insurance information, user geographic location information, providers' specialization expertise, historical treatment outcome of providers, patient preferences (e.g., language, gender), and/or, appointment urgency to generate the list of in-person consultation appointments.

If the VHS 105 receives a decision of a telemedicine care pathway, then the method 400 proceeds to a step 440. In a step 440, the VHS 105 generates a list of telemedicine consultation appointments with a healthcare provider. For example, the VHS 105 may consider user-reported symptoms, user insurance information, user geographic location information, providers' specialization expertise, historical treatment outcome of providers, patient preferences (e.g., language, gender), and/or appointment urgency to generate the list of telemedicine consultation appointments.

In a step 445, the VHS 105 automatically schedules an in-person consultation based on a user input. The user input may, for example, include a user selection of an in-person consultation with a particular healthcare provider from the generated list.

In a step 450, the VHS 105 automatically schedules a telemedicine consultation based on a user input. The user input may, for example, include a user selection of a telemedicine consultation with a particular healthcare provider from the generated list.

In a step 455, the VHS 105 may, for example, automatically transmit the physician report (e.g., physician report 135) to a healthcare provider with which consultation was scheduled.

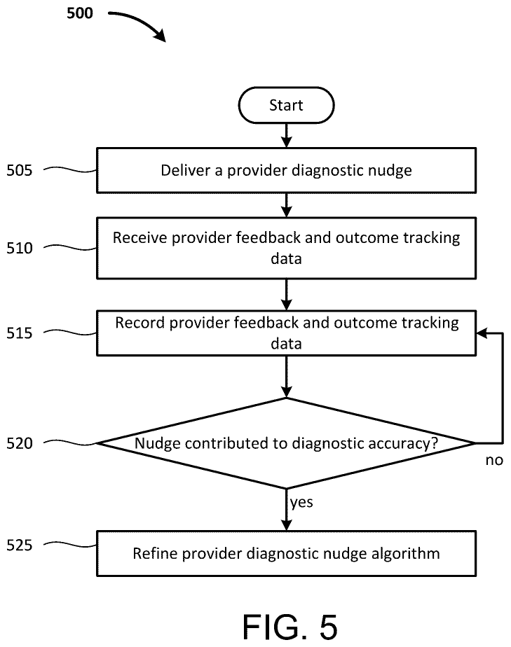

FIG. 5 is a flowchart illustrating an exemplary method 500 of an adaptive clinical decision support system with provider diagnostic nudges. For example, the method 500 may be performed by the VHS 105. In a step 505, the VHS 105 may, for example, transmit a provider diagnostic nudge to a healthcare provider's user device. The provider diagnostic nudge may, for example, be based on user-supplied physiological input data and a provider diagnostic nudge algorithm, such that the provider diagnostic nudge includes a suggested diagnostic and therapeutic recommendation. The provider diagnostic nudge may, for example, targeted suggestions designed to assist healthcare providers in considering relevant factors during the diagnostic process. The provider diagnostic nudge may, for example, advantageously prompt the healthcare provider to explore specific tests, alternative explanations, or rare but serious conditions based on the patient's symptoms and medical history. The provider diagnostic nudge may, for example, incorporate evidence-based guidance, such as references to clinical guidelines or weighted probability assessments of differential diagnoses. The provider diagnostic nudge may, for example, advantageously act as collegial reminders rather than directives, ensuring the physician's autonomy is respected. The provider diagnostic nudge may, for example, advantageously be tailored to the physician's workflow and aim to enhance diagnostic accuracy and efficiency while minimizing cognitive overload.

In a step 510, the VHS 105 may, for example, receive healthcare provider feedback and outcome tracking data. In a step 515, the VHS 105 may, for example, record provider feedback and outcome tracking data. For example, the VHS 105 may, for example, record provider feedback and outcome tracking data in the data store 250. For example, after a healthcare provider interacts with the VHS's 105 diagnostic nudges, their responses, such as whether a suggestion was helpful, dismissed, or acted upon, may be recorded. The VHS 105 may, for example, track patient outcomes, including confirmed diagnoses, treatment efficacy, and follow-up results, to evaluate the accuracy and utility of the nudges provided.

In a step 520, the recorded provider feedback and outcome data may, for example, be analyzed by the VHS 105 to determine whether the provider diagnostic nudge contributed to increasing diagnostic accuracy. For example, the VHS 105 may identify patterns, such as which nudges were most effective in specific clinical scenarios or which suggestions led to improved diagnostic accuracy. If no, the method 500 reverts to step 515.

If yes, the method 500 proceeds to a step 525. In a step 525, the VHS 105, may for example, refine its provider diagnostic nudge algorithm. For example, the VHS 105 may tailor future nudges to better align with real-world clinical needs and outcomes. This may, for example, advantageously create a dynamic learning loop, ensuring the nudge system evolves and becomes increasingly relevant and effective over time.

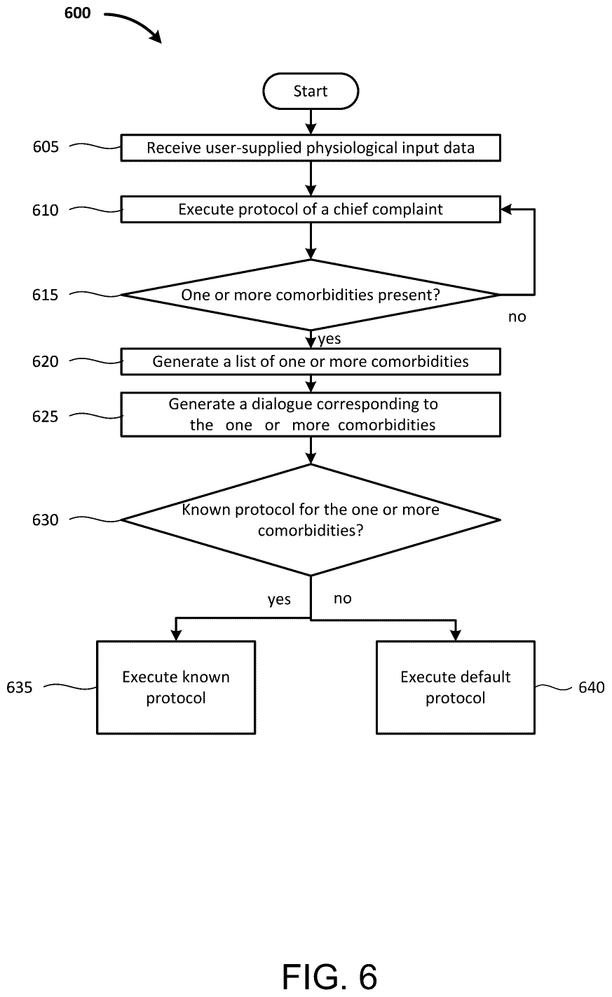

FIG. 6 is a flowchart illustrating an exemplary comorbidity agent method 600. For example, the method 600 may be performed by the VHS 105. In a step 605, the VHS 105 may, for example, receive user-supplied physiological input data. For example, the VHS 105 may generate auto-filled response fields based on predetermined protocols of a biomedical data processing system and receive user-supplied physiological input data from a user's interaction with the auto-filled response fields.

In a step 610, the VHS 105 may, for example, execute a protocol of a chief complaint of the user. For example, the VHS 105 may identify a primary issue the user is experiencing and perform a protocol. The protocol may, for example, include the steps of the methods 300 and 400.

In a step 615, the VHS 105 reaches a decision point where it decides whether a comorbidity is present in the patient based on user-supplied physiological input data. For example, the VHS 105 may identify any side complaints or additional conditions the patient may be experiencing alongside the chief complaint. If no, the method 600 reverts to step 610. If yes, the method 600 proceeds to a step 620.

In a step 620, the VHS 105 generates a list of one or more comorbidities. In a step 625, the VHS 105 may, for example, generate a dialogue corresponding to each of the one or more comorbidities. For example, the VHS 105 may generate a focused conversation about each additional condition or side complaint that a patient may have alongside their chief complaint.

In a step 630, the VHS 105 reaches a decision point where it decides for the one or more comorbidities whether there is known protocol for the one or more comorbidities in the VHS 105. For example, the VHS 105 may store predetermined protocols of the one or more comorbidities in the memory module 225. For example, the predetermined protocols may include auto-filled response fields corresponding to user-supplied physiological input data. If the VHS 105, decides yes, there is a known protocol for the one or more comorbidities in the VHS 105, then the method 600 proceeds to a step 635. In a step 635, the VHS 105 executes the known protocol. For example, the VHS 105 may generate auto-filled response fields corresponding to a medical a condition associated with the user-supplied physiological input data.

If the VHS 105 decides no, there is no there is no known protocol for the one or more comorbidities in the VHS 105, then the method 600 proceeds to a step 640. In a step 640, the VHS 105 executes a default protocol. The default protocol may, for example, include at least one of the following auto-filled response fields: (1) How long have you had that condition; (2) What symptoms do you have; (3) Have you ever had symptoms like this before? If yes, when; (4) Followed by, do the current symptoms feel the same? If yes, move to question #5. If no, how are the current symptoms different; and/or (5) Do you have any other symptoms you have not mentioned? If yes, go back to 4. If no, now go to our standard program and continue conversation.

Although various embodiments have been described with reference to the figures, other embodiments are possible.

In some aspects, the techniques described herein relate to a computer program product including: a program of instructions tangibly embodied on a computer readable medium wherein when the instructions are executed on a processor, the processor causes operations to be performed to automatically characterize individual health status and generate two or more tailored diagnostic outputs in a biomedical data processing system, the operations including: generate auto-filled response fields based on predetermined protocols of the biomedical data processing system; receive user-supplied physiological input data from a user's interaction with the auto-filled response fields; based on the user-supplied physiological input data, determine a response directive and a preliminary condition assessment including a determined response level of the response directive; and, generate two or more outputs based on the determined response directive and the determined preliminary condition assessment, such that the two or more outputs include content adapted to one or more intended output targets' expected comprehension levels.

In some aspects, the techniques described herein relate to a computer program product, wherein the one or more intended output targets' expected comprehension levels further include a technical user's expected comprehension level and a user's expected comprehension level.

In some aspects, the techniques described herein relate to a computer program product, wherein the two or more outputs further include a technical-facing output and a user-facing output.

In some aspects, the techniques described herein relate to a computer program product, wherein the operations further include: transmit the user-facing output to be displayed at a user device of a user.

In some aspects, the techniques described herein relate to a computer program product, wherein the operations further include: transmit the technical-facing output to be displayed at a user device of a technical user.

In some aspects, the techniques described herein relate to a computer program product, wherein the operations further include: generate a list of scheduled interactions with one or more designated data endpoints based on data from the biomedical data processing system's electronic data stores and timing modules; receive a selection input from the user of a selected interaction from the list of scheduled interactions with at least one of the one or more designated data endpoints; and, automatically transmit at least one of the two or more generated outputs to the one or more designated data endpoints that correspond to the selected interaction.

In some aspects, the techniques described herein relate to a computer program product, wherein the operations further include: retrieve a user profile including a user's location data, population attributes, condition history, and access parameters; and, apply the user profile to the determination of the response directive and the preliminary condition assessment, such that the response directive and the preliminary condition assessment are refined based on the user profile.

In some aspects, the techniques described herein relate to a computer program product, wherein the operations further include: determine whether the preliminary condition assessment exceeds a predetermined severity threshold; and based on the determination of whether the preliminary condition assessment exceeds a predetermined severity threshold, generate a routing directive to one or more designated data endpoints based on the user's location data and access parameters.

In some aspects, the techniques described herein relate to a computer program product, wherein the operations further include: prompt the user to choose a care pathway; based on a user-selected care pathway, generate a list of scheduled interactions with one or more designated data endpoints; receive a selection input from the user of a selected interaction from the list of scheduled interactions with at least one of the one or more designated data endpoints; and, automatically transmit at least one of the two or more generated outputs to the one or more designated data endpoints that correspond to the selected interaction.

In some aspects, the techniques described herein relate to a computer program product, wherein the operations further include: generate a provider diagnostic nudge based on the user-supplied physiological input data and a provider diagnostic nudge algorithm, wherein the provider diagnostic nudge includes a suggested diagnostic and therapeutic recommendation; transmit the provider diagnostic nudge to a user device of a technical user; receive technical user supplied feedback on the provider diagnostic nudge, wherein the technical user supplied feedback includes whether the diagnostic and therapeutic recommendation was followed; record in a data store the technical user supplied feedback; receive outcome tracking data, wherein the outcome tracking data includes medical data inputs of the user; and, record in the data store the outcome tracking data; determine whether the provider diagnostic nudge contributed to improved diagnostic accuracy and patient outcomes by analyzing the technical user supplied feedback and the outcome tracking data; and, if the provider diagnostic nudge is determined to contribute to improved diagnostic accuracy and patient outcomes, refine the provider diagnostic nudge algorithm to prioritize generating the provider diagnostic nudge to other users with similar user-supplied physiological input data as the user.

In some aspects, the techniques described herein relate to a computer program product, wherein the operations further include: based on the user-supplied physiological input data, determine whether the user has one or more comorbidities; generate a list the one or more comorbidities; generate auto-filled response fields for each of the one or more comorbidities based on predetermined protocols of the one or more comorbidities in the biomedical data processing system; receive user-supplied physiological input data from a user's interaction with the auto-filled response fields; based on the user-supplied physiological input data, determine a response directive and a preliminary condition assessment including a determined response level of the response directive; and, generate two or more outputs based on the determined response directive and the determined preliminary condition assessment, such that the two or more outputs include content adapted to one or more intended output targets' expected comprehension levels.

In some aspects, the techniques described herein relate to a system including: a data store including a program of instructions; and, a processor operably coupled to the data store such that, when the processor executes the program of instructions, the processor causes operations to be performed to automatically characterize individual health status and generate two or more tailored diagnostic outputs in a biomedical data processing system, the operations including: generate auto-filled response fields based on predetermined protocols of the biomedical data processing system; receive user-supplied physiological input data from a user's interaction with the auto-filled response fields; based on the user-supplied physiological input data, determine a response directive and a preliminary condition assessment including a determined response level of the response directive; and, generate two or more outputs based on the determined response directive and the determined preliminary condition assessment, such that the two or more outputs include content adapted to one or more intended output targets' expected comprehension levels.

In some aspects, the techniques described herein relate to a system, wherein the one or more intended output targets' expected comprehension levels further include a technical user's expected comprehension level and a user's expected comprehension level.

In some aspects, the techniques described herein relate to a system, wherein the two or more outputs further include a technical-facing output and a user-facing output.

In some aspects, the techniques described herein relate to a system, wherein the operations further include: transmit the user-facing output to be displayed at a user device of the user.

In some aspects, the techniques described herein relate to a system, wherein the operations further include: transmit the technical-facing output to be displayed at a user device of a technical user.

In some aspects, the techniques described herein relate to a system, wherein the operations further include: generate a list of scheduled interactions with one or more designated data endpoints based on data from the biomedical data processing system's electronic data stores and timing modules; receive a selection input from the user of a selected interaction from the list of scheduled interactions with at least one of the one or more designated data endpoints; and, automatically transmit at least one of the two or more generated outputs to the one or more designated data endpoints that correspond to the selected interaction.

In some aspects, the techniques described herein relate to a system, wherein the operations further include: retrieve a user profile including a user's location data, population attributes, condition history, and access parameters; and, apply the user profile to the determination of the response directive and the preliminary condition assessment, such that the response directive and the preliminary condition assessment are refined based on the user profile.

In some aspects, the techniques described herein relate to a system, wherein the operations further include: determine whether the preliminary condition assessment exceeds a predetermined severity threshold; and based on the determination of whether the preliminary condition assessment exceeds a predetermined severity threshold, generate a routing directive to one or more designated data endpoints based on the user's location data and access parameters.

In some aspects, the techniques described herein relate to a system, wherein the operations further include: prompt the user to choose a care pathway; based on a user-selected care pathway, generate a list of scheduled interactions with one or more designated data endpoints; receive a selection input from the user of a selected interaction from the list of scheduled interactions with at least one of the one or more designated data endpoints; and, automatically transmit at least one of the two or more generated outputs to the one or more designated data endpoints that correspond to the selected interaction.

Although various embodiments have been described with reference to the figures, other embodiments are possible.

Although an exemplary system has been described with reference to the figures, other implementations may be deployed in other industrial, scientific, medical, commercial, and/or residential applications.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IOT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A computer program product comprising:
a program of instructions tangibly embodied on a computer readable medium wherein when the instructions are executed on a processor, the processor causes operations to be performed to automatically characterize individual health status and generate two or more tailored diagnostic outputs in a biomedical data processing system, the operations comprising:
generate auto-filled response fields based on predetermined protocols of the biomedical data processing system;
receive user-supplied physiological input data from a user's interaction with the auto-filled response fields;
based on the user-supplied physiological input data, determine a response directive and a preliminary condition assessment comprising a determined response level of the response directive; and,
generate two or more outputs based on the determined response directive and the determined preliminary condition assessment, such that the two or more outputs include content adapted to one or more intended output targets' expected comprehension levels.

2. The computer program product of claim 1, wherein the one or more intended output targets' expected comprehension levels further comprise a technical user's expected comprehension level and a user's expected comprehension level.

3. The computer program product of claim 1, wherein the two or more outputs further comprise a technical-facing output and a user-facing output.

4. The computer program product of claim 3, wherein the operations further comprise:
transmit the user-facing output to be displayed at a user device of a user.

5. The computer program product of claim 3, wherein the operations further comprise:
transmit the technical-facing output to be displayed at a user device of a technical user.

6. The computer program product of claim 1, wherein the operations further comprise:
generate a list of scheduled interactions with one or more designated data endpoints based on data from the biomedical data processing system's electronic data stores and timing modules;
receive a selection input from the user of a selected interaction from the list of scheduled interactions with at least one of the one or more designated data endpoints; and,
automatically transmit at least one of the two or more generated outputs to the one or more designated data endpoints that correspond to the selected interaction.

7. The computer program product of claim 1, wherein the operations further comprise:
retrieve a user profile comprising a user's location data, population attributes, condition history, and access parameters; and,
apply the user profile to the determination of the response directive and the preliminary condition assessment,
such that the response directive and the preliminary condition assessment are refined based on the user profile.

8. The computer program product of claim 7, wherein the operations further comprise:
determine whether the preliminary condition assessment exceeds a predetermined severity threshold; and
based on the determination of whether the preliminary condition assessment exceeds a predetermined severity threshold, generate a routing directive to one or more designated data endpoints based on the user's location data and access parameters.

9. The computer program product of claim 1, wherein the operations further comprise:
prompt the user to choose a care pathway;
based on a user-selected care pathway, generate a list of scheduled interactions with one or more designated data endpoints;
receive a selection input from the user of a selected interaction from the list of scheduled interactions with at least one of the one or more designated data endpoints; and,
automatically transmit at least one of the two or more generated outputs to the one or more designated data endpoints that correspond to the selected interaction.

10. The computer program product of claim 1, wherein the operations further comprise:
generate a provider diagnostic nudge based on the user-supplied physiological input data and a provider diagnostic nudge algorithm, wherein the provider diagnostic nudge comprises a suggested diagnostic and therapeutic recommendation;
transmit the provider diagnostic nudge to a user device of a technical user;
receive technical user supplied feedback on the provider diagnostic nudge, wherein the technical user supplied feedback comprises whether the diagnostic and therapeutic recommendation was followed;
record in a data store the technical user supplied feedback;
receive outcome tracking data, wherein the outcome tracking data comprises medical data inputs of the user; and,
record in the data store the outcome tracking data;
determine whether the provider diagnostic nudge contributed to improved diagnostic accuracy and patient outcomes by analyzing the technical user supplied feedback and the outcome tracking data; and,
if the provider diagnostic nudge is determined to contribute to improved diagnostic accuracy and patient outcomes, refine the provider diagnostic nudge algorithm to prioritize generating the provider diagnostic nudge to other users with similar user-supplied physiological input data as the user.

11. The computer program product of claim 1, wherein the operations further comprise:
based on the user-supplied physiological input data, determine whether the user has one or more comorbidities;
generate a list the one or more comorbidities;
generate auto-filled response fields for each of the one or more comorbidities based on predetermined protocols of the one or more comorbidities in the biomedical data processing system;
receive user-supplied physiological input data from a user's interaction with the auto-filled response fields;
based on the user-supplied physiological input data, determine a response directive and a preliminary condition assessment comprising a determined response level of the response directive; and,
generate two or more outputs based on the determined response directive and the determined preliminary condition assessment, such that the two or more outputs include content adapted to one or more intended output targets' expected comprehension levels.

12. A system comprising:
a data store comprising a program of instructions; and,
a processor operably coupled to the data store such that, when the processor executes the program of instructions, the processor causes operations to be performed to automatically characterize individual health status and generate two or more tailored diagnostic outputs in a biomedical data processing system, the operations comprising:
generate auto-filled response fields based on predetermined protocols of the biomedical data processing system;
receive user-supplied physiological input data from a user's interaction with the auto-filled response fields;
based on the user-supplied physiological input data, determine a response directive and a preliminary condition assessment comprising a determined response level of the response directive; and,
generate two or more outputs based on the determined response directive and the determined preliminary condition assessment, such that the two or more outputs include content adapted to one or more intended output targets' expected comprehension levels.

13. The system of claim 12, wherein the one or more intended output targets' expected comprehension levels further comprise a technical user's expected comprehension level and a user's expected comprehension level.

14. The system of claim 12, wherein the two or more outputs further comprise a technical-facing output and a user-facing output.

15. The system of claim 14, wherein the operations further comprise:
transmit the user-facing output to be displayed at a user device of the user.

16. The system of claim 14, wherein the operations further comprise:
transmit the technical-facing output to be displayed at a user device of a technical user.

17. The system of claim 12, wherein the operations further comprise:
generate a list of scheduled interactions with one or more designated data endpoints based on data from the biomedical data processing system's electronic data stores and timing modules;
receive a selection input from the user of a selected interaction from the list of scheduled interactions with at least one of the one or more designated data endpoints; and,
automatically transmit at least one of the two or more generated outputs to the one or more designated data endpoints that correspond to the selected interaction.

18. The system of claim 12, wherein the operations further comprise:
retrieve a user profile comprising a user's location data, population attributes, condition history, and access parameters; and,
apply the user profile to the determination of the response directive and the preliminary condition assessment,
such that the response directive and the preliminary condition assessment are refined based on the user profile.

19. The system of claim 18, wherein the operations further comprise:
determine whether the preliminary condition assessment exceeds a predetermined severity threshold; and
based on the determination of whether the preliminary condition assessment exceeds a predetermined severity threshold, generate a routing directive to one or more designated data endpoints based on the user's location data and access parameters.

20. The system of claim 12, wherein the operations further comprise:
prompt the user to choose a care pathway;
based on a user-selected care pathway, generate a list of scheduled interactions with one or more designated data endpoints;
receive a selection input from the user of a selected interaction from the list of scheduled interactions with at least one of the one or more designated data endpoints; and,
automatically transmit at least one of the two or more generated outputs to the one or more designated data endpoints that correspond to the selected interaction.
